(19) United States
(12) Patent Application Publication
(10) Pub. No.: US 2026/0000477 A1
(43) Pub. Date: Jan. 1, 2026

(54) APPARATUS FOR POSITIONING OR NAVIGATING NEEDLES RELATIVE TO OBJECT, SYSTEM COMPRISING SAME, AND METHOD USING SAME

(71) Applicant: HEICor Biomedical Interventions GmbH, Heidelberg (DE)

(72) Inventors: Florian SICKLINGER, Heidelberg (DE); Florian LEUSCHNER, Heidelberg (DE)

(73) Assignee: HEICor Biomedical Interventions GmbH, Heidelberg (DE)

(21) Appl. No.: 19/252,375

(22) Filed: Jun. 27, 2025

(30) Foreign Application Priority Data
Jun. 27, 2024 (EP) 24 185 082.5

(51) Int. Cl. A61B 90/11; A61B 16/00; A61B 17/34; A61D 1/00

(52) U.S. Cl. CPC A61B 90/11; A61B 16/00; A61D 1/00; A61B 2017/3413

(57) ABSTRACT

There is provided an apparatus for positioning or navigating at least two needles (2) relative to an object, comprising at least two holding devices (3) for movably holding the needles (2), wherein the holding devices (3) each comprise an adjusting device (4) for moving the needles (2) independently of each other and relative to the object. Further, a system comprising such an apparatus and an imaging device (7) for monitoring and/or guiding a position of at least one needle (2) in the object and a method for induction of a heart failure in an animal with the use of such an apparatus or with the use of such a system are provided.

### FIELD OF THE INVENTION

The present invention relates to an apparatus for positioning or navigating at least two needles relative to an object, comprising at least two holding devices for movably holding the needles, wherein the holding devices each comprise an adjusting device for moving the needles independently of each other and relative to the object.

Further, the present invention relates to a system comprising such an apparatus and a method for induction of heart failure, HF, in an animal with the use of such an apparatus or with the use of such a system.

### BACKGROUND OF THE INVENTION

Heart failure remains to be a leading cause of death worldwide. Accordingly, there is an immense scientific and clinical need for a better understanding of acute and long-term changes in HF. The use of rodent myocardial infarction, MI, models is an established pillar of basic research investigating such processes and how to treat them. One of the most used models for induction of HF in mice is the transient ligation of the left anterior descending artery, LAD, during open-chest surgery. Even though investigations in this model have led to numerous crucial findings, LAD ligation is a low-throughput procedure with significant surgery-related mortality. Moreover, intubation, mechanical ventilation and thoracotomy within the procedure cause additional tissue damage and trigger a systemic inflammatory response which can confound the immune response evoked by MI. Additionally, the conventional method with opening of the chest is very time-intensive and low throughput. In addition, it is very stressful for the mouse and requires e.g. intensive analgetic regime after the procedure. All available closed-chest models are still suture- and ligation-based which means that the ligation must be performed manually. This is highly dependent on the skills of the surgeon which can result in low reproducibility and high variability. In addition, since the approach requires two steps, the procedure is even more time-intensive.

It is an object of the present invention to provide an apparatus, a system and a method for an effective and reproducible occlusion, partial or total, of a vessel of an animal by simple means.

In accordance with the invention and according to claim 1, the aforementioned object is accomplished by an apparatus for positioning or navigating at least two needles relative to an object, comprising at least two holding devices for movably holding the needles, wherein the holding devices each comprise an adjusting device for moving the needles independently of each other and relative to the object.

Further and according to claim 13, the aforementioned object is accomplished by a system comprising such an apparatus and an imaging device for monitoring and/or guiding a position of at least one needle in the object.

Further and according to claim 15, the aforementioned object is accomplished by a method for induction of heart failure in an animal, preferably with the use of an apparatus according to any one of claims 1 to 12 or with the use of a system according to claim 13 of 14, comprising the following steps: moving two needles to a coronary vessel of the animal; and occluding the coronary vessel, partial or total, by moving the needles together.

According to the invention it has been recognized that it is possible to solve the aforementioned object by providing a suitable apparatus specifically designed for a very effective and reproducible occlusion, partial or total, of a vessel of an animal. Such an apparatus is designed for allowing navigation of at least two needles. The apparatus comprises at least two holding devices for movably holding the needles, wherein the holding devices each comprise an adjusting device for moving the needles independently of each other and relative to the object. The needles are movable independently of each other and relative to the object, so that a reproducible occlusion of coronary vessels in mice for ischemia/reperfusion injury induction is possible by moving the needles to a coronary vessel of the animal and occlusing the coronary vessel by moving the needles together.

Thus, on the basis of the invention an effective and reproducible occlusion, partial or total, of a vessel of an animal by simple means is provided.

It has to be noted, that the term “needle” is used in this document in a very generic sense, so that the term “needle” also comprises cannulas, hollow needles, and tubes, for example.

For providing a very flexible moving of the needles the adjusting device can be designed for translatory moving of each needle in one, two or three spatial directions.

Further, under consideration of an even more flexible moving of the needles the adjusting device can be designed for rotary moving of each needle about one, two or three axes of rotation.

For providing a simplified moving of the needles the adjusting device can be designed for translatory moving of at least two needles together in one, two or three spatial directions.

Regarding a flexible and comfortable use of the apparatus the adjusting device can comprise an adjusting element or a main manipulator for a coarse moving and an adjusting element or a main manipulator for a fine moving for translatory moving one needle or at least two needles together in at least one spatial direction and/or for rotary moving a needle about at least one axis of rotation. Thus, a necessary adjustment of one or more needles can be performed quickly, even if the adjustment comprises a wide translatory and/or rotary movement in a first step and a fine adjustment in a second step.

For providing a compact apparatus which is easy to handle the holding devices can be coupled with or integrated into a base unit or manipulator. Thus, multiple holding devices can be handled together by means of such a base unit or manipulator.

In view of an effective use of the apparatus and a simple handling of the object the apparatus can further comprise an object platform for placement of the object, for example an animal, onto the object platform. The object platform can be easily placed in a necessary position with regard to the base unit or the manipulator.

Within a further embodiment, the apparatus can further comprise a ground platform onto which the holding devices, the base unit, the manipulator and/or the object platform can be positioned. Such a ground platform provides an individual positioning of the holding devices, the base unit, the manipulator and/or the object platform relative to each other.

With regard to a flexible and reliable positioning of the holding devices, the base unit, the manipulator and/or the object platform on the ground platform, the holding devices, the base unit, the manipulator and/or the object platform can be fixed on the ground platform by means of a magnetic stand or magnetic coupling. Such a kind of fixation is flexible and provides a locking of the holding devices, the base unit, the manipulator and/or the object platform by magnetic force. The magnetic force can be individually selected by an individual design of the magnetic stand or magnetic coupling, so that the holding devices, the base unit, the manipulator and/or the object platform can be easily positioned by a user. Alternatively, the base unit or the manipulator can comprise a clamping mechanism for clamping the base unit or the manipulator to the ground platform. The clamping mechanism can comprise a screw mechanism for easily fixing the base unit or the manipulator to the ground platform.

Within a further embodiment the ground platform can comprise a moving device for a translatory moving of the ground platform in a horizontal plane in an x-direction and/or in a y-direction, wherein the moving device can comprise a ground platform manipulator. Such a translatory moving of the ground platform allows relative position change between the ground platform and an imaging device. Since an object platform and a base unit or needle manipulator can be fixed to the ground platform, moving or navigation of the ground platform does not change the position of the needles within an animal during this moving or navigation.

In a further embodiment the apparatus can comprise multiple ground platforms, which can be designed for a side-by-side arrangement of the ground platforms. Thus, multiple ground platforms can easily be used together or in a platform-after-platform mode.

The ground platform can be designed in a size which allows multiple holding devices, base units, manipulators and/or object platforms to be positioned on the ground platform. Thus, an effective and time-saving use of the apparatus is possible.

The imaging device for monitoring and/or guiding a position of at least one needle in the object can comprise an ultrasound transducer, which can be held by a holding equipment.

Exemplary advantages and aspects of embodiments of the present invention are given below:

According to an embodiment a specifically designed manipulator or micromanipulator is provided which allows high-precision navigation of at least two needles in parallel. The manipulator or micromanipulator in combination with a specific object platform and/or ground platform enables reproducible occlusion of coronary vessels in mice for ischemia/reperfusion injury induction.

In order to induce ischemia reperfusion in mice, the left coronary artery can be visualized using an imaging device operating for example with ultrasound. In a minimal-invasive approach, two needles attached to the manipulator or micromanipulator are inserted into the closed chest under image guidance and navigated to the target site of the coronary vessels. Afterwards, the needles are placed either ventral, needle 1, or dorsal, needle 2, of the coronary artery followed by occlusion of the vessel by moving the needles together. The manipulator or micromanipulator allows high-precision navigation of the two needles in all three dimensions inside the chest. Moreover, the angulation of the needles in all dimensions can be modified. These feature are important to guarantee optimal occlusion and adjustments throughout the procedure. After a defined time, which can be up to 2 hours, the needles are removed, for reperfusion.

There are several ways how to design and further develop the teaching of the present invention in an advantageous way. To this end it is to be referred to the following explanation of examples of embodiments of the invention, illustrated by the drawing.

### BRIEF DESCRIPTION OF THE DRAWINGS

FIG. 1 shows a perspective view of an embodiment of an apparatus and system according to the invention.

FIG. 2 shows a perspective view of the apparatus shown in FIG. 1.

FIG. 3 shows a further perspective view of the apparatus shown in FIG. 1 indicating the possibility of a needle rotation about a y-axis.

FIG. 4 shows a further perspective view of the apparatus shown in FIG. 1 indicating the possibility of a needle rotation about a z-axis.

FIG. 5 shows a further perspective view of the apparatus shown in FIG. 1 indicating the possibility of a needle translation in an x-axis and a z-axis.

FIG. 6 illustrates the echocardiography-guided induction of myocardial ischemia/reperfusion (EG-IR) in mice. FIG. 6A shows representative ultrasound B-mode and color Doppler images of the left anterior descending artery during stepwise vessel occlusion using micromanipulator-controlled needles. Electrocardiogram (ECG) tracings recorded in parallel show ST-segment elevation confirming successful occlusion. FIG. 6B is a schematic depiction of a high-throughput setup with multiple animal imaging stations. A single ultrasound transducer is shared across platforms to facilitate parallel EG-IR procedures. FIG. 6C presents quantified serum levels of cardiac troponin T (cTnT) 24 hours after a 45-minute EG-IR procedure. FIGS. 6D-6E show Masson Trichrome-stained heart sections and the corresponding quantification of infarct size as a percentage of left ventricular circumference 14 days after injury. FIG. 6F illustrates flow cytometric quantification of CD45⁺ leukocytes in cardiac tissue 2 days post-EG-IR. FIGS. 6G-6H provide quantification of extracellular matrix remodeling via immunofluorescence staining for periostin (Postn) and collagen 1a2 (Col1a2) at days 7 and 14 post-injury. FIG. 6I shows representative immunofluorescence images of cardiac sections stained for Postn, wheat germ agglutinin (WGA), and DAPI.

FIG. 7 presents echocardiographic assessment of cardiac function and area-at-risk (AAR) in mice subjected to EG-IR. FIGS. 7A-7C display longitudinal echocardiographic measurements of left ventricular ejection fraction (LVEF), end-diastolic volume (EDV), and global longitudinal strain (GLS) following either 30- or 60-minute ischemia protocols.

FIG. 7D shows quantification of AAR size based on echocardiographic assessment during ischemia. FIG. 7E is a scatter plot showing correlation between AAR size and serum cTnT levels at 24 hours post-injury. FIG. 7F depicts a representative B-mode image used to define the AAR based on regional wall akinesia. FIG. 7G presents myocardial wall thickness in the AAR upon reperfusion. FIG. 7H is a scatter plot correlating wall thickness with cTnT levels. FIG. 7I shows a B-mode ultrasound image highlighting edema formation and backscatter enhancement in the AAR during reperfusion.

FIG. 8 compares the efficiency and systemic effects of EG-IR with conventional IR models (OC-IR and CC-IR). FIG. 8A shows hands-on procedure time for each IR model. FIG. 8B illustrates recovery times measured as the interval between procedure completion and free ambulation.

FIG. 8C presents Kaplan-Meier survival curves over a 28-day follow-up period. FIG. 8D tracks post-procedural body weight changes. FIGS. 8E-8F show flow cytometric analysis of neutrophils and macrophages in cardiac tissue 4 days after IR. FIGS. 8G-8H quantify circulating blood neutrophils and Ly6Chigh monocytes. FIGS. 8I-8L report myocardial concentrations of chemokines CXCL1, CXCL2, CXCL5, and CCL2.

FIG. 9 demonstrates equivalence in functional outcomes and reproducibility between EG-IR and CC-IR models. FIG. 9A shows representative B-mode echocardiographic images taken during the ischemic period for both IR models. FIG. 9B quantifies AAR size using long-axis measurements. FIG. 9C shows bull's-eye plots of regional wall motion 4 weeks post-injury. FIG. 9D displays segmental wall motion score indices (SWMSI) at 1 and 4 weeks. FIGS. 9E-9G present longitudinal changes in LVEF, EDV, and GLS after IR. FIG. 9H-9J compare AAR size and 28-day LVEF outcomes across three independent research centers, demonstrating interinstitutional reproducibility.

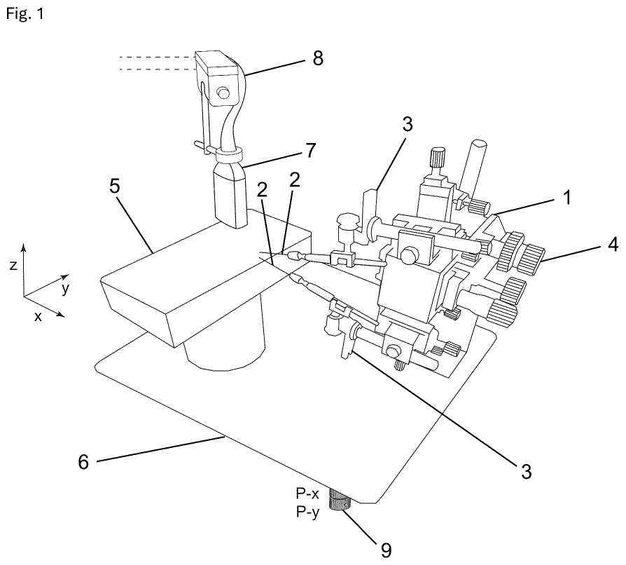

FIG. 1 shows a perspective view of an embodiment of an apparatus in the form of a manipulator 1. The manipulator 1 comprises two needles 2 which are movably held by individual holding devices 3. Each holding device 3 comprises an adjusting device 4 for moving the needles 2 independently of each other and relative to an object platform 5.

The manipulator 1 and the object platform 5 are positioned on a ground platform 6. An imaging device 7 comprising an ultrasound transducer is also positioned on the ground platform 6. An object, for example an animal, can be positioned on the object platform 5. The ground platform 6 can be moved in a horizontal x-y-plane.

The manipulator 1 is a specialized micro-manipulator which allows high-precision navigation of two needles 2. The micro-manipulator enables combined translation of both needles 2 in all three dimensions and isolated navigation of each needle independently, i.e. translation in two dimensions and rotation in two axes.

The object platform 5 is a specialized animal or rodent platform which allows placement of a sedated animal in supine position. Both, the animal or object platform 5 and the manipulator 1 are attached to the ground platform 6. By the use of magnetic stands, the position of the animal or object platform 5 and the manipulator 1 is flexible, but can be locked.

The system includes a holding equipment 8 which allows placement of an imaging device 7 in the form of an ultrasound transducer relative to an object or animal. The holding equipment 8 is not connected to the ground platform 6 and can be positioned/navigated independently from the ground platform 6.

As indicated in FIG. 1, the ground platform 6 can be moved by employment of a platform manipulator 9 or micro-manipulator, P-x for translation on the x-axis and P-y for translation on the y-axis. Navigation of the ground platform 6 allows relative position change between the ground platform 6, including animal or object platform 5 and needle manipulator 1, and the imaging device 7. Since the animal or object platform 5 and the needle manipulator 1 are attached to the ground platform 6, navigation of the ground platform 6 does not change the position of the needles 2 within an object or animal during the intervention.

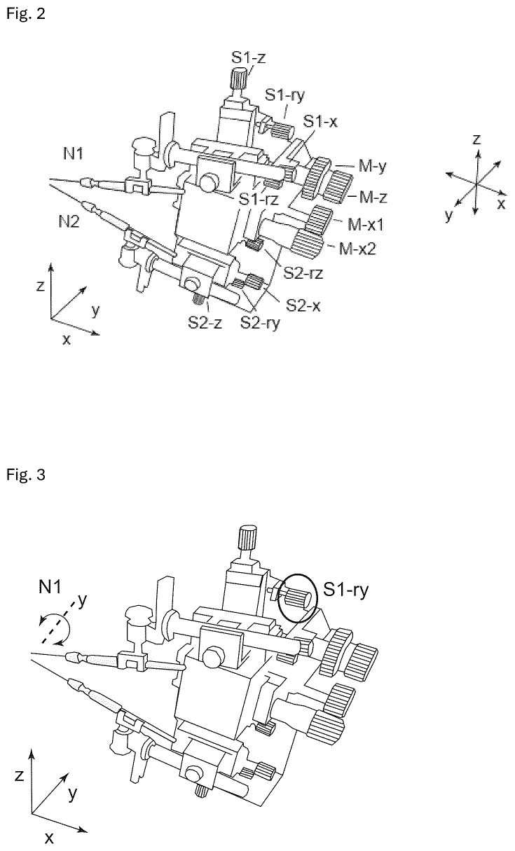

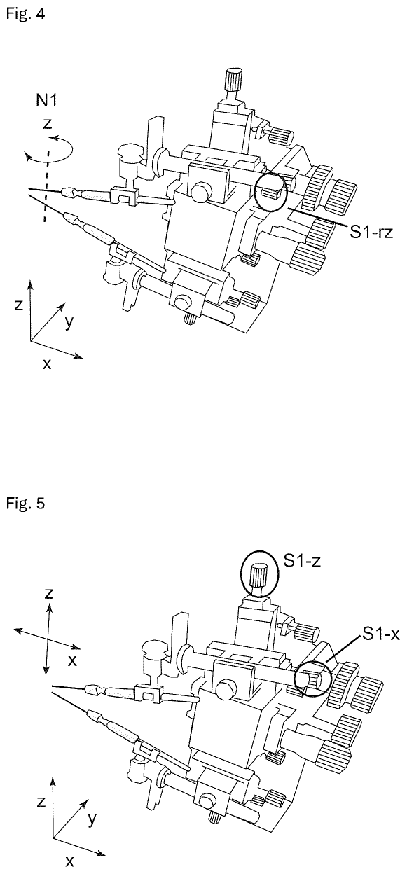

FIG. 2 shows a perspective view of the apparatus shown in FIG. 1 indicating the multiple moving possibilities of the needles 2. FIG. 3-5 show further perspective views of the apparatus shown in FIG. 1, particularly indicating the possibility of a needle rotation about a y-axis and about a z-axis—FIG. 3 and FIG. 4—and the possibility of a needle translation in an x-axis and a z-axis-FIG. 5.

FIG. 2-5 show Cartesian coordinate axes x, y and z for explaining the possibilities of moving the needles 2.

In the following, the two needles 2 are specified by the reference signs N1 and N2.

Further, the adjusting device 4 comprises adjusting elements in the form of main manipulators M-y, M-z, M-x1 and M-x2. For movement of the needles N1 and N2, the main manipulators M-y, M-z, M-x1 and M-x2 allow translation of both needles N1 and N2 at the same time. M-x1 is for coarse translational movement on the x-axis, whereas M-x2 enables fine adjustment of translational movement on the x-axis.

The adjusting device 4 comprises further adjusting elements in the form of four further manipulators S1-z, S1-x, S1-ry and S1-rz and four further manipulators S2-z, S2-x, S2-ry and S2-rz for movement of each individual needle N1 and N2, respectively.

For movement of needle N1, the following four further manipulators can be adjusted:
S1-z allows translation of needle N1 in the z-axis.
S1-x allows translation of needle N1 in the x-axis.
S1-ry allows rotation of needle N1 around the y-axis, xz-plane.
S1-rz allows rotation of needle N1 around the z-axis, xy-plane.

For movement of needle N2, the following four further manipulators can be adjusted:
S2-z allows translation of needle N2 in the z-axis.
S2-x allows translation of needle N2 in the x-axis.
S2-ry allows rotation of needle N2 around the y-axis, xz-plane.
S2-rz allows rotation of needle N2 around the z-axis, xy-plane.

According to further embodiments of the invention an arrangement of multiple intervention ground platforms 6 side by side is possible. Thereby, a single operator can efficiently utilize waiting times during vessel occlusion and perform interventions in parallel. Embodiments include either arrangement of multiple ground platforms 6 side by side, with an attached animal or object platform 5 and micromanipulator or manipulator 1 on each ground platform 6, or multiple animal or object platforms 5 and micromanipulators or manipulators 1 positioned on one for example big ground platform 6.

The present invention further relates to a method for inducing myocardial ischemia in a living animal using a minimally invasive approach that allows for controlled, reversible occlusion of a coronary vessel. The method is particularly suited for use in small laboratory animals, such as mice and rats, and facilitates real-time monitoring of the procedure through the use of an imaging device.

Accordingly, in one aspect, the invention provides a method for inducing myocardial ischemia in an anesthetized animal, comprising the steps of:
(a) placing the anesthetized animal in a supine position on a support surface;
(b) visualizing a coronary vessel of the animal using an imaging device;
(c) inserting a first needle and a second needle into the animal such that the first needle is positioned ventral and the second needle is positioned dorsal of the coronary vessel; and
(d) advancing the first and second needles toward each other to occlude the coronary vessel between the needles.

In preferred embodiments, the method is carried out in a mouse or a rat, and most preferably in a mouse. The procedure may be performed without the need for opening the chest cavity (i.e., through a closed-chest approach), thereby reducing procedural trauma and animal mortality. The first and second needles are inserted percutaneously and navigated to the target region of the coronary vessel through the intact thoracic wall.

The positioning of the needles is preferably performed under the guidance of an imaging device (7), such as a high-resolution ultrasound system capable of B-mode and/or color Doppler imaging. This allows for real-time visualization of the coronary vasculature, guidance of needle insertion, and confirmation of both vessel location and occlusion status.

In some embodiments, the imaging device is further used to monitor the position and movement of at least one of the needles within the thoracic cavity during insertion and occlusion. Continuous or intermittent imaging may be applied throughout the procedure to ensure precise needle alignment and to avoid damage to adjacent structures such as the lungs or left atrial appendage.

The occlusion step comprises placing one needle ventral and the other needle dorsal to the target coronary vessel, then advancing the needles toward each other in a coordinated manner such that the vessel is compressed and flow is blocked. The angulation and spacing of the needles may be optimized under imaging control to ensure complete and consistent occlusion, while minimizing the risk of vessel rupture or myocardial perforation.

The duration of coronary occlusion may vary depending on the experimental goal. In some embodiments, the occlusion is maintained for a period ranging from 1 second to 3 hours, and in preferred embodiments, for 30 minutes to 90 minutes. These timeframes allow for the modeling of a range of ischemic conditions, including short-term reversible ischemia and prolonged ischemia with significant myocardial injury.

Upon completion of the ischemia phase, the needles may be withdrawn to permit reperfusion of the previously occluded vessel, and restoration of flow can be confirmed via imaging (e.g., reappearance of Doppler signal) and/or ECG normalization.

The method described herein offers several advantages over traditional open-chest ischemia models:
It is minimally invasive, requiring no thoracotomy or mechanical ventilation;
It provides real-time control over the occlusion and reperfusion process;
It supports high reproducibility in infarct size and localization;
It improves animal welfare and reduces recovery time; and
It is compatible with high-throughput experimental designs by enabling rapid turnover and parallel preparation of multiple animals.

This method is particularly useful in cardiovascular research for investigating myocardial ischemia/reperfusion injury, inflammatory responses, fibrotic remodeling, cardiac function decline, and pharmacological interventions.

### EXAMPLES

### Example 1: Animal Preparation and Anesthesia Protocol

C57BL/6J and C57BL/6N mice aged 8 to 20 weeks were used for all procedures. Animals were anesthetized via inhalation of 2% isoflurane (induction) and maintained at 1.5% isoflurane during interventions. Subcutaneous injection of buprenorphine (0.05 mg/kg) was administered pre-operatively for analgesia.

Mice were placed in the supine position on a heated ultrasound imaging platform (VisualSonics, Toronto, Canada), and body temperature was continuously monitored and maintained at 36-37° C. using a rectal probe. Adequate anesthesia depth was confirmed by absence of rear foot reflexes. Respiratory rate was kept above 50/min throughout the procedure.

### Example 2: Visualization of the Coronary Artery and Vessel Occlusion

The left anterior descending (LAD) coronary artery was identified by transthoracic echocardiography using a high-frequency ultrasound probe (MS550D or MX550D, VisualSonics) in modified parasternal short-axis view with color Doppler mode.

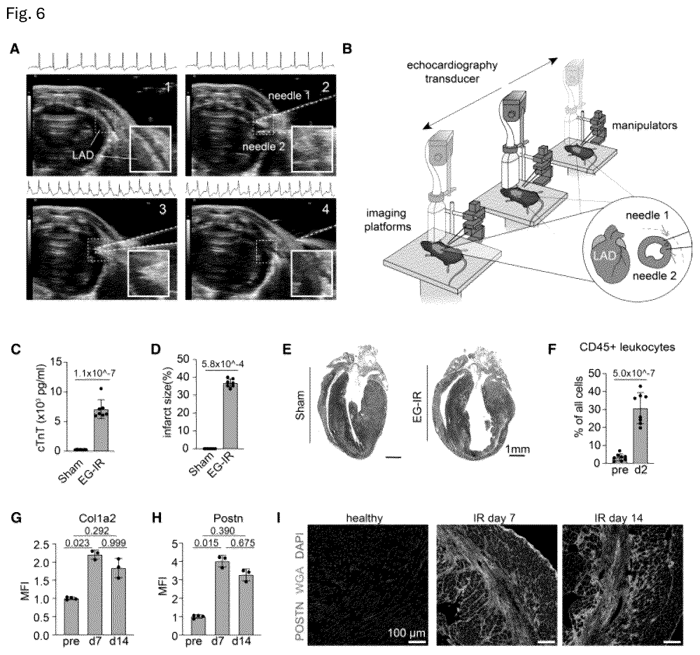

Two 30-gauge hypodermic needles, mounted on a custom 5-axis micromanipulator (HEICor BMI GmbH, Heidelberg, Germany), were inserted through the closed left thoracic wall and positioned ventral and dorsal to the LAD under real-time imaging. The needles were moved simultaneously to compress and occlude the artery. Successful occlusion was confirmed by:
Absence of distal blood flow in Doppler mode,
Regional wall akinesia in the area-at-risk (AAR), and
ST-segment elevation in ECG recordings. (FIG. 6A).

### Example 3: Induction of Ischemia and Reperfusion

After confirming successful occlusion, ischemia was maintained for 30, 45, 60, or 120 minutes. Reperfusion was initiated by retracting the needles. Re-establishment of blood flow was confirmed by Doppler ultrasound and normalization of ECG parameters.

For sham controls, both needles were inserted to the epicardial surface but no occlusion was applied.

In high-throughput settings, up to three imaging stations were operated in parallel using a single ultrasound probe moved between micromanipulator platforms. A multi-channel isoflurane delivery system allowed simultaneous anesthesia maintenance for multiple animals. (FIG. 6B).

### Example 4: Cardiac Function Analysis

Cardiac function was assessed at multiple time points using Vevo 2100 or 3100 systems. Left ventricular ejection fraction (LVEF), end-diastolic volume (EDV), and global longitudinal strain (GLS) were derived from long-axis and short-axis views.

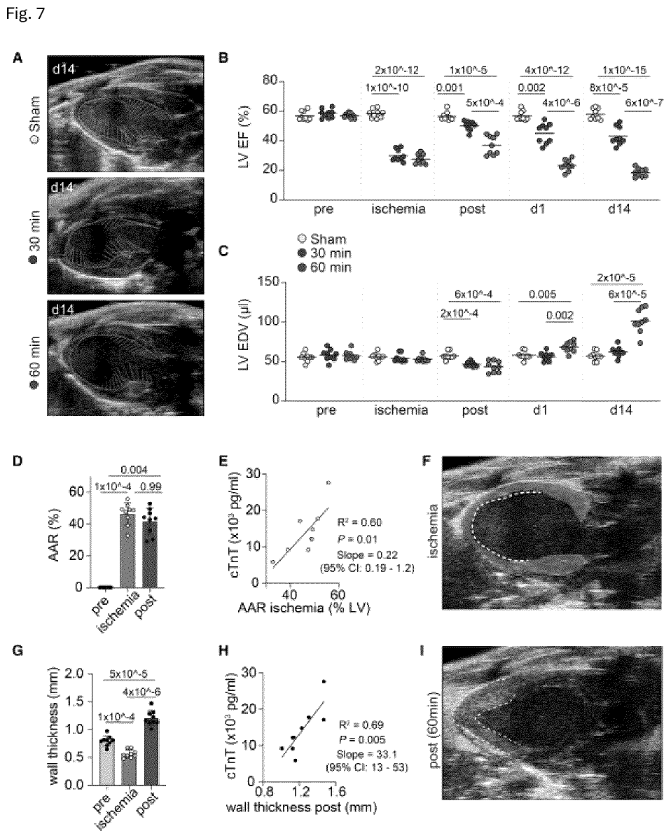

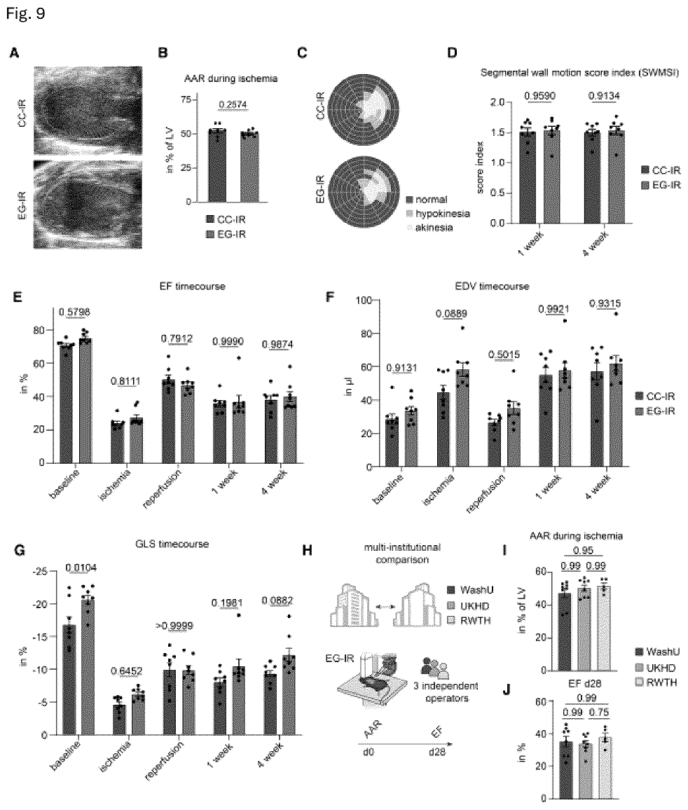

Regional function was quantified using a 12-segment scoring system (normal=1, hypokinetic=2, akinetic=3), and wall motion score index (WMSI) was calculated. (FIGS. 7A-7C, 9C-9D). AAR was measured intraprocedurally based on echocardiographic akinesia and correlated with serum cardiac troponin T (cTnT) levels at 24 hours post-procedure. (FIGS. 7D-7F).

Myocardial wall thickness and backscatter upon reperfusion were assessed as markers of edema formation and correlated with myocardial injury. (FIGS. 7G-7I).

### Example 5: Histological and Molecular Confirmation of Injury

At defined time points post-IR, hearts were harvested, fixed in 4% paraformaldehyde for 3 hours, cryopreserved in 30% sucrose, and embedded in O.C.T. compound. 9 μm cryosections were stained with Masson's Trichrome for infarct size analysis.

Fibrosis was quantified by immunostaining for Postn and Col1a2. Flow cytometry was used to analyze leukocyte infiltration. (FIGS. 6C-6I).

Serum cTnT was significantly increased after 45-minute EG-IR compared to sham. Immunofluorescence confirmed ECM remodeling and fibroblast activation.

### Example 6: Comparison to Conventional Models (OC-IR and CC-IR)

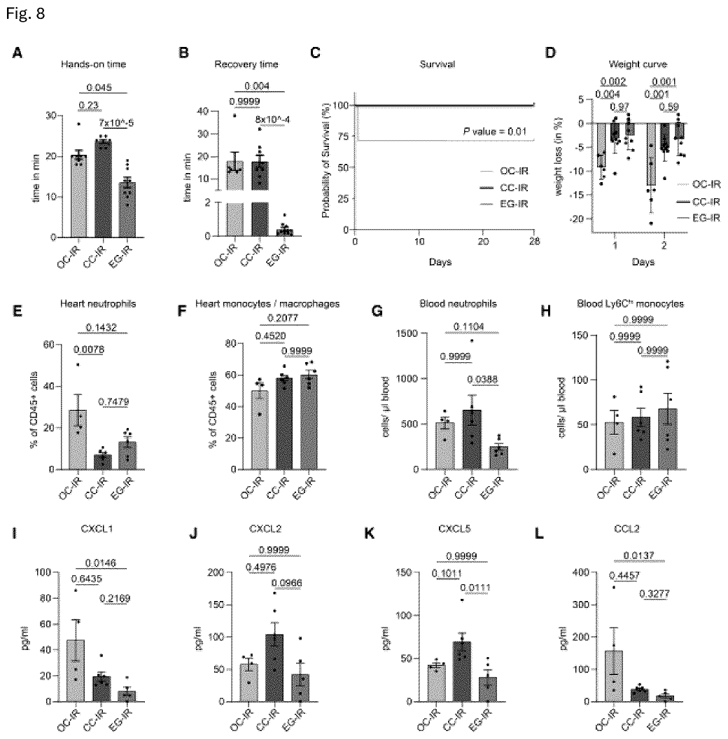

Echocardiography-guided IR (EG-IR) was compared to open-chest (OC-IR) and closed-chest (CC-IR) surgical models. EG-IR exhibited:
Shorter hands-on time (FIG. 8A),
Faster recovery (FIG. 8B),
Lower mortality (FIG. 8C),
Less post-procedural weight loss (FIG. 8D), and
Reduced neutrophil infiltration and chemokine expression (FIGS. 8E-8L).

Despite reduced invasiveness, EG-IR induced infarct size and cardiac dysfunction comparable to CC-IR, as shown by segmental wall motion analysis and functional follow-up. (FIGS. 9A-9G).

### Example 7: Multi-Institutional Reproducibility

The EG-IR method was replicated at multiple research centers (Heidelberg, Washington University, RWTH Aachen), achieving consistent results in:
Area-at-risk size during ischemia,
LVEF decline after 28 days,
Procedural success rates (>95%). (FIGS. 9H-9J).

The method was validated in both sexes and in older mice, with a total of over 250 procedures yielding less than 3% mortality.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### LIST OF REFERENCE SIGNS

1 manipulator
2 needle
3 holding device
4 adjusting device
5 object platform
6 ground platform
7 imaging device
8 holding equipment
9 platform manipulator
M-y main manipulator
M-z main manipulator
M-x1 main manipulator
M-x2 main manipulator
N1 needle
N2 needle
S1-z further manipulator
S1-x further manipulator
S1-ry further manipulator
S1-rz further manipulator
S2-z further manipulator
S2-x further manipulator
S2-ry further manipulator
S2-rz further manipulator

1. An apparatus for positioning or navigating at least two needles relative to an object, comprising at least two holding devices for movably holding the needles, wherein the holding devices each comprise an adjusting device for moving the needles independently of each other and relative to the object.

2. The apparatus according to claim 1, wherein the adjusting device is designed for translatory moving of each needle in one, two or three spatial directions.

3. The apparatus according to claim 1, wherein the adjusting device is designed for rotary moving of each needle about one, two or three axes of rotation.

4. The apparatus according to claim 1, wherein the adjusting device is designed for translatory moving of at least two needles together in one, two or three spatial directions.

5. The apparatus according to claim 1, wherein the adjusting device comprises an adjusting element or a main manipulator for a coarse moving and an adjusting element or a main manipulator for a fine moving for translatory moving one needle or at least two needles together in at least one spatial direction and/or for rotary moving a needle about at least one axis of rotation.

6. The apparatus according to claim 1, wherein the holding devices are coupled with or integrated into a base unit or manipulator.

7. The apparatus according to claim 1, wherein the apparatus further comprises an object platform for placement of the object, for example an animal, onto the object platform.

8. The apparatus according to claim 1, wherein the apparatus further comprises a ground platform onto which the holding devices, the base unit, the manipulator and/or the object platform can be positioned.

9. The apparatus according to claim 8, wherein the holding devices, the base unit, the manipulator and/or the object platform are or is fixed on the ground platform by means of a magnetic stand or magnetic coupling.

10. The apparatus according to claim 8, wherein the ground platform comprises a moving device for a translatory moving of the ground platform in a horizontal plane in an x-direction and/or in a y-direction, wherein the moving device can comprise a ground platform manipulator.

11. The apparatus according to claim 8, wherein the apparatus comprises multiple ground platforms, which are designed for a side-by-side arrangement of the ground platforms.

12. The apparatus according to claim 8, wherein multiple holding devices, base units, manipulators and/or object platforms are positioned on the ground platform.

13. A system comprising an apparatus according to claim 1 and an imaging device for monitoring and/or guiding a position of at least one needle in the object.

14. The system according to claim 13, wherein the imaging device comprises an ultrasound transducer, which can be held by a holding equipment.

15. A method for induction of a heart failure in an animal, preferably with the use of an apparatus according to claim 1, comprising the following steps:
moving two needles to a coronary vessel of the animal; and
occluding the coronary vessel, partial or total, by moving the needles together.

16. A method for induction of a heart failure in an animal, preferably with the use of a system according to claim 13, comprising the following steps:
moving two needles to a coronary vessel of the animal; and
occluding the coronary vessel, partial or total, by moving the needles together.

17. The method according to claim 15, wherein the animal is a mouse or a rat.

18. The method according to claim 15, wherein the two needles are inserted into the closed chest of the animal and navigated to the target site of the coronary vessel.

19. The method according to any one of claim 15, wherein the step of occluding the coronary vessel comprises placing one needle ventral and the other needle is placed dorsal of the coronary vessel.

20. The method according to any one of claim 15, wherein the imaging device is used for monitoring the position of at least one needle in the animal and/or for guiding the movement of at least one needle in the animal.

21. The method according to any one of claim 15, wherein the occluding comprises optimizing the angulation and the relative position of the needles to guarantee optimal occlusion.

22. The method according to any one of claim 15, wherein occluding occurs for a period of 1 second to 3 h.

23. The method according to any one of claim 15, wherein occluding occurs for a period of 30 min to 90 min.
